(19) United States
(12) Patent Application Publication
(10) Pub. No.: US 2008/0045523 A1
(43) Pub. Date: Feb. 21, 2008

(54) Synthesis of Polyoxygenated Nitrogen Systems, Comprising Reactions Between Enamines of 1,3-Dioxan-5-Ones and Nitroolefins

(75) Inventors: Ricardo Alonso Alonso, Santiago de Compostela (ES); Lidia Ozores Viturro, Santiago de Compostela (ES); Fernando Cagide Fagin, Santiago de Compostela (ES); Juan Carlos Ortiz Lara, Santiago de Compostela (ES)

Correspondence Address: INTELLECTUAL PROPERTY / TECHNOLOGY LAW, PO BOX 14329, RESEARCH TRIANGLE PARK, NC, 27709, US

(73) Assignee: Universidade de Santiago de Compostela, Santiago de Compostela (ES)

(21) Appl. No.: 11/572,707

(22) PCT Filed: Jun. 20, 2005
(86) PCT No.: PCT/ES05/00374

§ 371 Date: Feb. 20, 2007

(30) Foreign Application Priority Data
Jun. 25, 2004 (ES) P200401547

(51) Int. Cl. C07D 319/06; A61K 31/341; A61K 31/357; A61K 31/5377; C07D 413/02; A61P 31/00; C07D 307/02

(52) U.S. Cl. 514/231.5; 514/452; 514/471; 544/148; 549/397; 549/496

(57) ABSTRACT

The invention relates to the synthesis of polyoxygenated nitrogen systems, comprising reactions between enamines of 1,3-dioxan-5-ones and nitroolefins. More specifically, the invention relates to the reaction between enamines having formula II which are derived from 1,3-dioxan-5-ones I and nitroolefins having formula III. In this way, novel polyoxygenated nitrogen systems are generated having formula IV and V, which, as they are or following simple transformations, constitute synthetic intermediates and/or analogues of different systems of proven biological/pharmacological interest, such as certain antibiotics, or tetrodotoxin, pancratistatin or the analogues thereof. The aforementioned reactions involve the use of enamines II in Michael-type addition reactions and in direct annealing processes with double acceptor systems at relative positions 1, 3. Said N acceptors include nitroolefins III, wherein R═CHO (alphanitroenals). The invention also relates to the preparation thereof, which is based on the oxidation of the corresponding precursor alcohols VII, and the use of same in annealing processes.

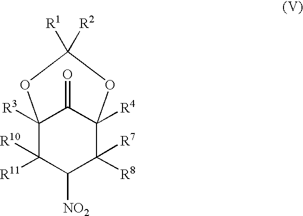

### FIELD OF THE INVENTION

The invention is included in the general area of development of new processes for the stereocontrolled synthesis of polyoxygenated nitrogen systems and particularly in the area of development of new synthetic strategies for the preparation of those compounds having polyoxygenated nitrogen cyclohexane systems as a structural subunit, as is the case of: a) certain antibiotics such as streptomycin, kanamycins, fortimicins or hygromycin A, b) certain compounds blocking voltage-dependent sodium channels such as tetrodotoxin and the analogues thereof, and c) certain compounds with a proven antitumor and antiviral efficacy such as pancratistatin and the analogues thereof.

State of the Art

Polyoxygenated nitrogen cyclohexane systems constitute an essential part of the structures of a high number of compounds of great biological and pharmacological interest. These compounds include:

1. Certain antibiotics such as streptomycin, kanamycins, fortimicins or hygromycin A:

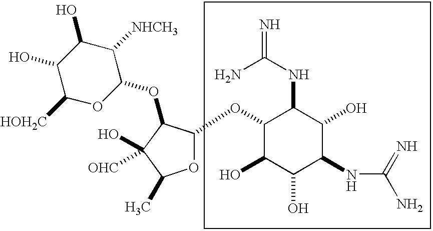

Streptomycin

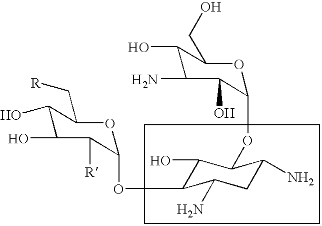

| | R | R′ |
|---|---|---|
| Kanamycin A | NH₂ | OH |
| Kanamycin B | NH₂ | NH₂ |
| Kanamycin C | OH | NH₂ |

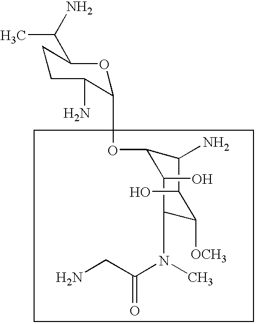

Fortimicin A

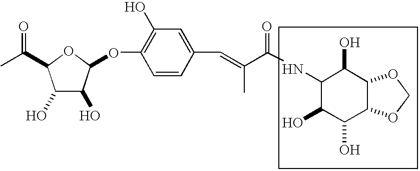

Hygromycin A

2. Certain compounds blocking voltage-dependent sodium channels (and therefore blocking nervous impulse and pain sensation) such as tetrodotoxin and the analogues thereof:

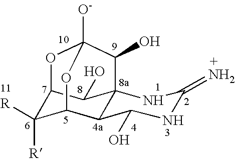

| | R′ | R |
|---|---|---|
| Tetrodotoxin (TTX) | OH | CH₂OH |
| 11-deoxy-TTX | OH | CH₃ |
| 6-epi-TTX | CH₂OH | OH |
| 11-oxo-TTX | OH | CHO |
| 11-nor-TTX-6-(R)-ol | H | OH |
| 11-nor-TTX-6(S)-ol | OH | H |
| Chiriquiioxin | OH | CH(OH)CH(NH₂)CO₂H |
| | | (R) (S) |
| 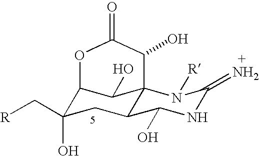 | | |

| | R | R′ |
|---|---|---|
| 1-hydroxy-5,11-dideoxy-TTX | H | OH |
| 5-deoxy-TTX | OH | H |
| 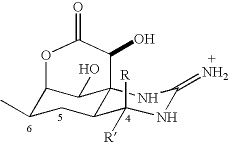 | | |

| | R | R′ |
|---|---|---|
| 5,6,11-trideoxy-TTX | H | OH |
| 4-epi-5,6,11-trideoxy-TTX | OH | H |

and 3. Certain compounds with a proven antitumor and antiviral efficacy such as pancratistatin and the analogues thereof.

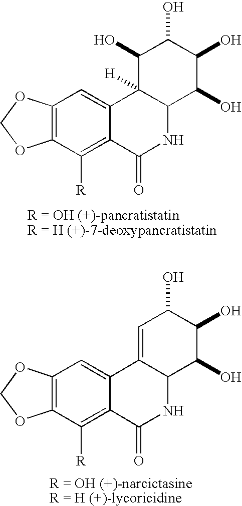

In spite of the importance of the mentioned polyoxygenated nitrogen systems derived from their presence as structural subunits in compounds of great interest, as illustrated above, the efficient access to said systems is not currently solved. By way of example, the following facts are enough to demonstrate this:
1. The synthesis of the polyhydroxylated aminocyclohexane of the antibiotic hygromycin A recently described by Trost (Trost, B. M.; Dudash, J., Jr.; Hembre, E. J., Chem. Eur. J. 2001, 7, 1619-1629) still requires a total of 13 steps, despite the fact that it involves an important improvement compared to the only synthesis described previously (Chida, N.; Ohtsuka, M.; Nakazawa, K.; Ogawa, S., J. Org. Chem. 1991, 56, 2976-2983).
2. The total synthesis of tetrodotoxin in an enantiomerically pure form has just been achieved by two groups (Hinman, A.; Du Bois, J., J. Am. Chem. Soc. 2003, 125, 11510-11511, Ohyabu, N.; Nishikawa, T.; Isobe, M., J. Am Chem. Soc. 2003, 125, 8798-8805) and in both cases requires a considerably high number of steps, more than 30 in the first case and more than 70 in the second.
3. Although up to eight total syntheses of pancratistatin have currently been described, none of them has solved the problem for supplying this compound, a problem which has determined the standstill of preclinical studies which were being conducted in the U.S. National Cancer Institute (NCI) (see the last total syntheses described by S. Kim et al.: Ko, H.; Kim, E.; Park, J. E.; Kim, D.; Kim, S., J. Org. Chem. 2004, 69, 112-121, as well as the synthesis studies by Kornienko et al.: Nadein, O. N.; Kornienko, A. Org. Lett. 2004, 6, 831-834 and the references mentioned therein).

From the foregoing, it is obvious that there is a need to invent new, general synthetic strategies allowing access to polyoxygenated nitrogen systems, particularly cyclohexane systems, in a small number of steps.

This patent sets forth a solution allowing the expeditious access to polyoxygenated nitrogen systems starting from simple and easily obtainable compounds, in a convergent manner and in a very reduced number of steps.

### DESCRIPTION OF THE INVENTION

The present invention describes the formation of polyoxygenated nitrogen systems by means of the reaction of enamines having formula II with nitroolefins having formula III.

Enamines II can be prepared by the reaction of ketones I, which obviously allow an extraordinarily varied substitution for the indicated substituents R¹-R⁴, with nitrogen compounds having formula R⁵R⁶NH, wherein R⁵ and R⁶ can also have extraordinarily different values (scheme 1, example 1). The reaction of enamines II with nitroolefins III, where the only possible structural limitation could be that in which any of the substituents R⁷ or R⁸ introduces a double bond conjugated with the one in the nitroolefin, leads to polyoxygenated nitrogen compounds having formula IV by Michael-type addition (Scheme 1, example 2).

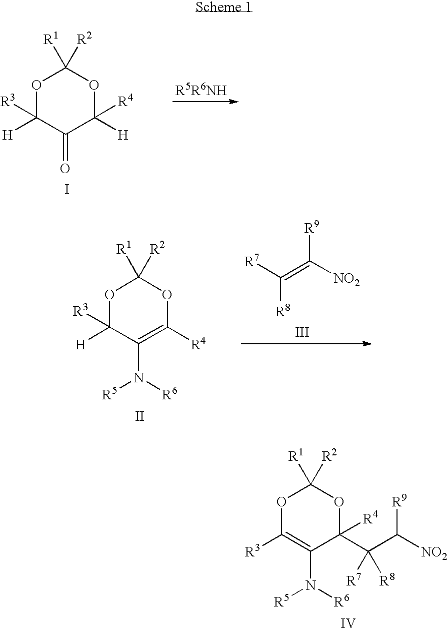

When nitroolefins III have:
i) either an electrophilic R⁹ group which can be attacked by an enamine, such as III wherein R⁹═C(O)X wherein X can be a hydrogen or a halogen atom or O, S, Se or Te atoms joined to an alkyl, aryl or acyl group, or such as III wherein R⁹═C[=M(CO)₅]X wherein M can be Cr, Mo or W, and X can be an O or N atom joined to one or more hydrogen atoms or to alkyl, aryl or acyl groups, or
ii) an electrophilic center precursor R⁹ group such as III wherein R⁹═CXYZ, wherein X and Y can be hydrogen or halogen atoms or alkyl or aryl groups which can in turn be substituted by one or several identical or different substituents selected from halogen atoms, alkyl, aryl, hydroxy, alkoxy, aryloxy, amino, alkylamino or arylamino groups, and X is any leaving group such as: a) a halogen atom, or b) an —OC(O)R^{b} group, or
c) an —OS(O)ₙR^{b} wherein n can have the values 1 or 2, and R^{b} can be a linear, branched or cyclic alkyl group or an aryl group, which groups can in turn be substituted by one or several identical or different substituents selected from halogen atoms, alkyl, hydroxy, alkoxy, aryloxy, amino or alkylamino groups, then the reaction of these nitroolefins III with enamines II does not stop in compound IV, but the enamino group present in IV reacts with the acceptor group in R⁹ to form the intermediates A which lead to bicyclic polyoxygenated nitrogen compounds having formula V by hydrolysis (scheme 2, examples 3, 4 and 5).

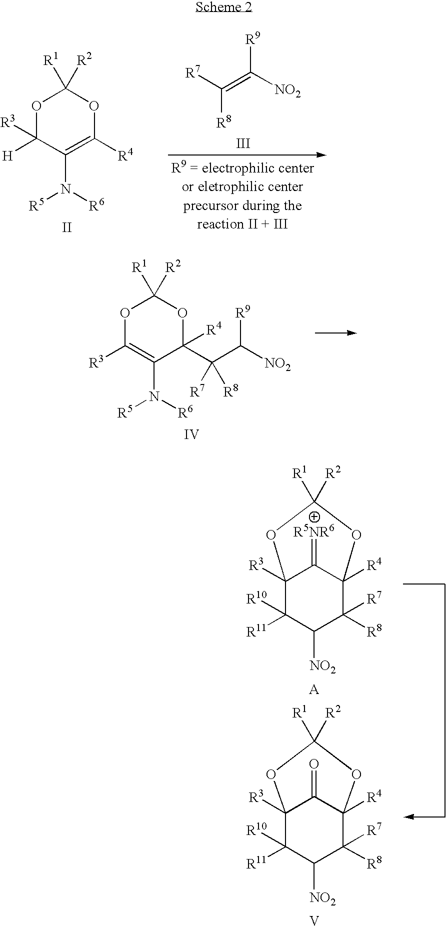

Alternatively, bicycles V can be obtained starting from compounds having formula IV by means of a sequence including: a) an addition of compounds IV to compounds such as R¹⁰R¹¹C=0, which leads to compounds having formula VI, b) the conversion of compounds having formula VI to compounds having formula VII by i) conversion of the hydroxy group of VI into a good leaving group such as a halogen atom or an ester derived from a carboxylic acid or from a sulfonic acid by the reaction with acylation or sulfonylation agents, followed by ii) elimination of the leaving group, and c) cyclization of VII by means of an intramolecular Michael-type addition (scheme 3, example 6+7).

The conversion of compounds having formula IV into compounds having formula V can be carried out in practice by both isolating one or several of the intermediates obtained in each step (examples 6+7), and by carrying out all the steps successively, one after the other, in the same reactor without isolating the intermediate products (example 8).

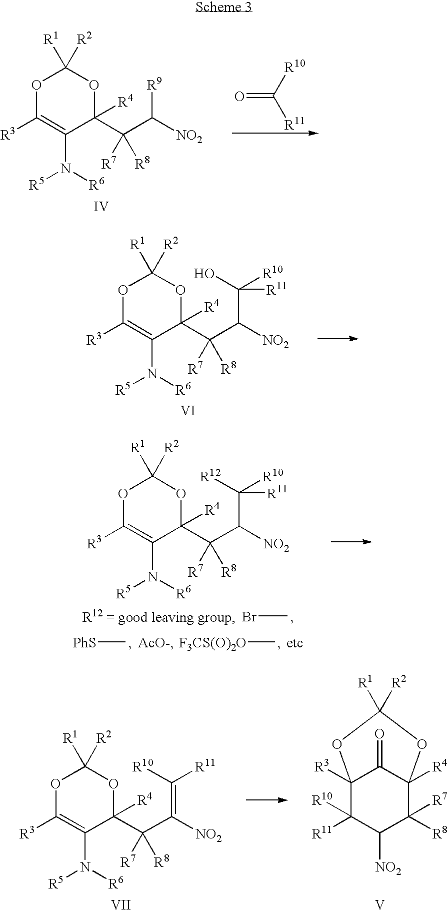

The novel and very convenient preparation of nitroolefins III having a group R⁹=CHO by means of the oxidation of their corresponding precursor alcohols having formula VIII (Scheme 4, example 9) must also be emphasized.

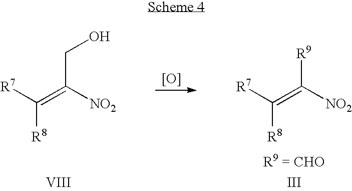

It is evident from their structural examination that both the compounds of type IV and those of type V are extraordinarily versatile precursors of other, very diverse polyoxygenated nitrogen systems into which they can be transformed by means of the suitable combination of simple reactions. In fact, several of these transformations of the systems IV and V have been successfully explored, guided by their synthetic use as precursors of the polyoxygenated nitrogen systems referred to in the introduction: certain antibiotics, tetrodotoxin and the analogues thereof, and pancratistatin and the analogues thereof. In practice, this confers an extraordinary added value to the synthetic methodology claimed herein as a route for accessing said systems IV and V and to the derivatives thereof by extension.

### EXAMPLES

The following examples are included below only for the purpose of contributing to a better understanding of the invention. The examples are by no means limited to the field of application of the described invention.

### Example 1

### Preparation of Enamine IIa Starting from Ketone Ia

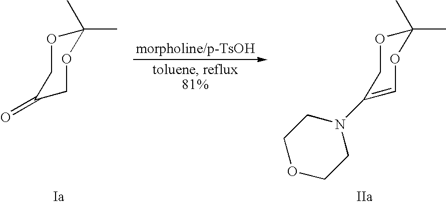

A solution of ketone Ia (4.12 g, 31.64 mmol), morpholine (3.3 mL, 37.97 mmol) and p-toluenesulfonic acid (307 mg) in toluene (250 mL, 1.9 M) was subjected to reflux for 5 h with azeotropic elimination of water by means of a Dean-Stark. The solvent was eliminated under reduced pressure and the raw product was redissolved in Et₂O, washed with a saturated NaHCO₃ solution, dried over anhydrous Na₂SO₄ and concentrated in a rotary evaporator. After a fast filtration through neutral alumina (activity I) (AcOEt-hexane 20:80), enamine IIa (5.08 g, 81%) was obtained as a colorless oil.

¹H-NMR (CDCl₃, 250 MHz, TMS) δ: 5.99 (t, J=1.3 Hz, 1H, CH), 4.20 (d, J=1.3 Hz, 2H, CH₂), 3.74-3.70 (m, 4H, 2×CH₂), 2.70-2.66 (m, 4H, 2×CH₂), 1.45 (s, 6H, 2×CH₃).

¹³C-NMR and DEPT (63 MHz) δ: 125.8 (C), 125.5 (CH), 97.9 (C), 66.7 (2×CH₂), 59.3 (CH₂), 50.0 (2×CH₂), 24.0 (2×CH₃).

MS (low resolution EI) m/z (%): 239 (M⁺, 0.03), 141 (2), 58 (100).

MS (low resolution CI⁺) m/z (%): 201 (M⁺+2, 16), 200 (M⁺+1, 80), 199 (M⁺, 55), 170 (59), 143 (37), 142 (100), 141 (enal generated by the retro-hetero-Diels-Alder reaction of the dioxenone ring, 91), 112 (42), 59 (73).

### Example 2

### Preparation of IVa Starting from Enamine IIa and Nitroolefin IIIa

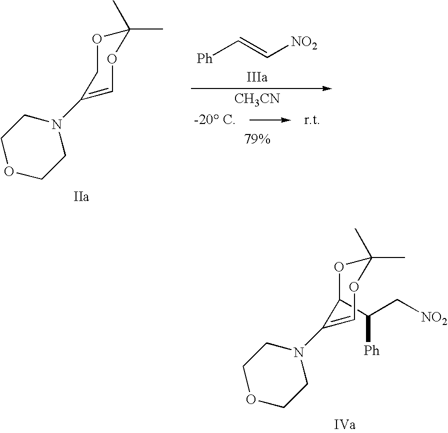

Nitroolefin IIIa (441 mg, 2.90 mmol) dissolved in acetonitrile was added to a solution of enamine IIa (583 mg, 2.90 mmol) in dry acetonitrile (1.5 mL) under argon at −20° C. The reaction was followed by means of thin layer chromatography (AcOEt-hexane 20:80) and after 12 h at room temperature (r.t.) the disappearance of the starting substance was observed. The reaction mixture was diluted with Et₂O and washed with water. The organic phase was dried with anhydrous Na₂SO₄ and the purification of the obtained residue by means of column chromatography (AcOEt-hexane, 10:90) led to polyoxygenated nitrogen system IVa (796 mg, 79%).

Spectroscopic Data of IVa in CDCl₃:

¹H-NMR (CDCl₃, 250 MHz, TMS) δ: 7.39-7.35 (m, 2H, ArH), 7.25-7.22 (m, 3H, ArH), 5.76 (s, 1H, CH), 4.94 (dd, J=13.5, J=8.5, 1H, CH₂NO₂), 4.67 (dd, J=13.5, J=6.9, 1H, CH₂NO₂), 4.66 (d, J=2.2, 1H, CH), 3.99 (ddd, J=8.5, J=6.9, J=2.8, 1H, CHAr), 3.72-3.68 (m, 4H, 2×CH₂), 2.75-2.67 (m, 2H, CH₂), 2.23-2.15 (m, 2H, CH₂), 1.47 (s, 3H, CH₃), 1.44 (s, 3H, CH₃).

¹³C-NMR and DEPT (63 MHz) δ: 135.8 (Ar), 129.6 (CH+ArH), 127.7 (ArH), 127.5 (ArH), 126.6 (C), 98.1 (C), 76.8 (CH₂NO₂), 67.7 (CH), 66.8 (2×CH₂), 50.5 (2×CH₂), 45.1 (CHAr), 27.7 (CH₃), 20.3 (CH₃).

MS (low resolution EI) m/z (%): 348 (M⁺, 1), 290 (α,β-unsaturated ketone generated by the retro-hetero-Diels-Alder reaction of the dioxenone ring, 4), 244 (16), 230 (18), 156 (100), 126 (60), 115 (28), 91 (27), 77 (15).

Spectroscopic Data of IVa in CO(CD₃)₂:

¹H-NMR (CO(CD₃)₂, 250 MHz, TMS) δ: 7.37-7.33 (m, 2H, ArH), 7.16-7.07 (m, 3H, ArH), 5.68 (s, 1H, CH), 4.92 (dd, J=13.5, J=6.6, 1H, CH₂NO₂), 4.81 (dd, J=13.2, J=4.1, 1H, CH₂NO₂), 4.73 (d, J=2.8, 1H, CH), 3.95 (ddd, J 9.1, J=6.6, J=2.8, 1H, CHAr), 3.58-3.55 (m, 4H, CH₂), 2.74-2.64 (m, 2H, CH₂), 2.10-2.02 (m, 2H, CH₂), 1.33 (s, 3H, CH₃), 1.30 (s, 3H, CH₃).

¹³C-NMR and DEPT (63 MHz) δ: 138.2 (C), 131.8 (CH), 130.9 (CH), 129.2 (CH), 128.9 (CH), 128.8 (C), 99.5 (C), 79.3 (CH₂NO₂), 70.2 (CH), 68.2 (2×CH₂), 52.4 (2×CH₂), 47.1 (CHAr), 29.0 (CH₃), 21.6 (CH₃).

### Example 3

### Preparation of the Bicyclic Polyoxygenated Nitrogen Compound Va by the Reaction of Enamine IIb and Nitroolefin IIIb

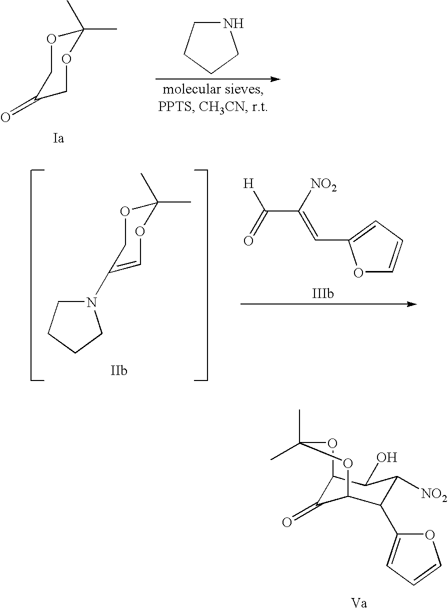

A solution of ketone Ia (50 mg, 0.38 mmol), pyrrolidine (39 μl, 0.46 mmol), molecular sieves (100 mg) and (catalytic) PPTS in dry acetonitrile (4 mL, 0.11 M) under argon was stirred for 40 min at r.t. Nitroolefin IIIb (71 mg, 0.42 mmol) was added to the reaction mixture at 0° C. containing the enamine IIb thus prepared, and after 40 min at 0° C. a mixture of 0.1 M HCl/acetone (5:20) was added. After 1 h at 0° C., the reaction mixture was washed with an NH₄Cl solution, and was extracted with Et₂O. The organic phase was dried over anhydrous Na₂SO₄, concentrated in a rotary evaporator and the raw product was purified by means of column chromatography (AcOEt-hexane 10:90), Va being obtained (51 mg, 45%).

### Example 4

### Preparation of Bicyclic Polyoxygenated Nitrogen Compounds Va and Vb and of Nitrogen Systems IVb and IVc by the Reaction of Enamine IIa and Nitroolefin IIIb

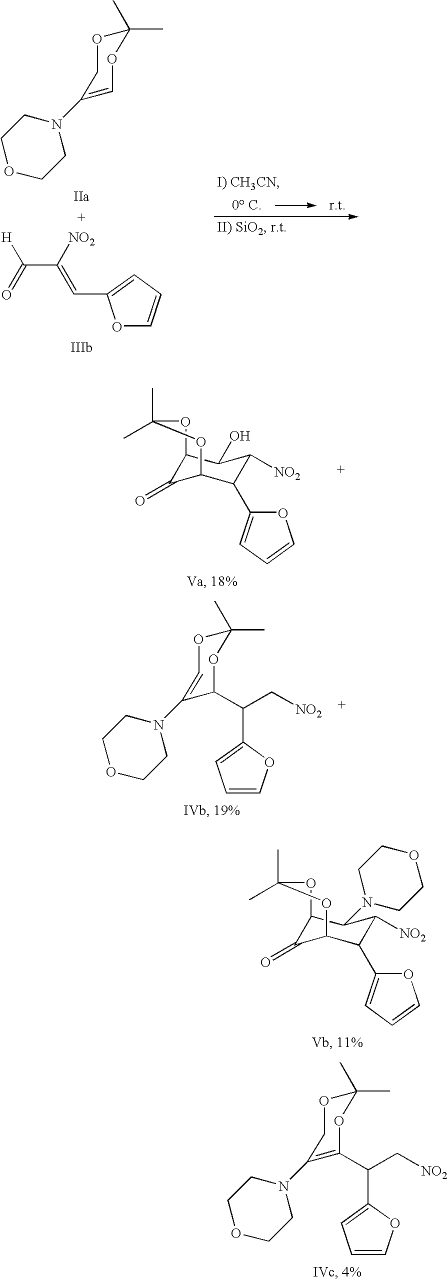

Nitroolefin IIIb (423 mg, 2.61 mmol) was added to a solution of enamine IIa (520 mg, 2.61 mmol) in acetonitrile (13 mL, 0.2 M) under argon at 0° C. After stirring for half an hour at 0° C. and 4 h at r.t., SiO₂ was added and was left stirring for 2 days at r.t. After evaporating the solvent, column chromatography (AcOEt-hexane 10:90) led to bicycles Va (139 mg, 18%) and Vb (102 mg, 11%), to the trisubstituted enamine IVb (172 mg, 19%) and to its isomeric tetrasubstituted enamine IVc (39 mg, 4%).

Spectroscopic Data of Bicycle Va:

¹H-NMR (CDCl₃, 300 MHz, TMS) δ: 7.38 (d, J=1.9, 1H, CH), 6.39 (d, J=3.4, 1H, CH), 6.35 (dd, J=3.4, J=1.9, 1H, CH), 5.33 (dd, J=11.6, J=9.4, 1H, CHNO2), 4.55 (m, 1H, CH), 4.51 (m, 1H, CH), 4.20 (dd, J=9.4, J=1.3, 1H, CHOH), 3.51 (dd, J=11.6, J=1.3, 1H, CH), 3.01 (d, J=10.7, 1H, OH), 1.57 (s, 3H, CH₃), 1.51 (s, 3H, CH₃).

¹³C-NMR and DEPT (63 MHz) δ: 205.0 (CO), 147.1 (C), 143.0 (CH), 110.7 (CH), 108.9 (CH), 99.9 (C), 89.0 (CHNO₂), 79.1 (CH), 77.0 (CH), 76.2 (CH), 44.4 (CH), 28.3 (CH₃), 25.5 (CH₃).

MS (low resolution EI) m/z (%): 282 (17), 250 (24), 193 (57), 163 (50), 123 (100), 81 (80), 59 (69).

IR (KBr): 1739 (medium, narrow, CO), 1555 (intense, narrow, NO₂) cm⁻¹.

Spectroscopic Data of Bicycle Vb:

¹H-NMR (CDCl₃, 250 MHz, TMS) δ: 7.33 (s, 1H, CH), 6.36 (s, 1H, CH), 6.30 (s, 1H, CH), 5.60 (t, J=11.0, 1H, CHNO₂), 4.77 (s, 1H, CH), 4.44 (s, 1H, CH), 3.67-3.49 (m, 5H, CH+2×CH₂), 3.13 (d, J=11.0, 1H, CH), 3.00-2.94 (m, 2H, CH₂), 2.60-2.53 (m, 2H, CH₂), 1.48 (s, 3H, CH₃), 1.42 (s, 3H, CH₃).

¹³C-NMR and DEPT (100 MHz) δ: 206.2 (CO), 147.8 (C), 142.8 (CH), 110.6 (CH), 108.7 (CH), 99.0 (C), 83.6 (CHNO₂), 77.3 (CH), 76.3 (CH), 72.5 (CH), 67.2 (2×CH₂), 50.2 (2×CH₂), 45.6 (CH), 28.2 (CH₃), 24.7 (CH₃).

MS (low resolution EI) m/z (%): 366 (M⁺, 2), 262 (12), 237 (4), 192 (100), 158 (42), 121 (84), 83 (41).

Spectroscopic Data of Trisubstituted Enamine IVb:

¹H-NMR (CDCl₃, 250. MHz, TMS) δ: 7.28 (m, 1H, CH), 6.27 (m, 1H, CH), 6.21 (d, J=3.6, 1H, CH), 5.90 (s, 1H, CH), 4.84-4.66 (m, 2H, CH₂NO₂), 4.64 (m, 1H, CH), 4.19 (J=11.0, J=7.3, J=3.6, 1H, CH), 3.62-3.58 (m, 4H, 2×CH₂), 2.67-2.59 (m, 2H, CH₂), 2.35-2.26 (m, 2H, CH₂), 1.42 (s, 6H, 2×CH₃).

¹³C-NMR and DEPT (63 MHz) δ: 149.9 (C), 141.4 (CH), 129.7 (CH), 126.5 (C), 110.4 (CH), 107.7 (CH), 98.3 (C), 74.3 (CH₂NO₂), 67.0 (CH), 66.7 (2×CH₂), 50.6 (2×CH₂), 39.8 (CH), 27.7 (CH₃), 20.3 (CH₃).

MS (Low resolution EI) m/z (%): 338 (M⁺, 7), 280 (6), 234 (54), 220 (21), 156 (100), 128 (70), 94 (76), 65 (38).

Spectroscopic Data of Tetrasubstituted Enamine IVc:

¹H-NMR (CDCl₃, 250 MHz, TMS) δ: 7.31 (s, 1H, CH), 6.29 (d, J=3.6, 1H, CH), 6.14 (d, J=3.6, 1H, CH), 5.49 (dd, J=9.1, J=5.5, 1H, CH), 4.81 (dd, J=12.8, J=9.1, 1H, CH₂NO₂), 4.67 (dd, J=12.8, J=5.5, 1H, CH₂NO₂), 4.24 (s, 2H, CH₂), 3.73-3.70 (m, 4H, 2×CH₂), 2.75-2.62 (m, 4H, 2×CH₂), 1.40 (s, 6H, 2×CH₃).

¹³C-NMR and DEPT (75 MHz) δ: 150.5 (C), 141.7 (CH), 129.7 (C), 121.6 (C), 110.4 (CH), 106.7 (CH), 99.0 (C), 74.5 (CH₂NO₂), 67.4 (2×CH₂), 56.0 (CH₂), 51.4 (2×CH₂), 36.1 (CH), 24.7 (CH₃), 23.1 (CH₃).

### Example 5

### Preparation of Bicyclic Polyoxygenated Nitrogen Compound Vc by the Reaction of Enamine IIb and Nitroolefin IIIc

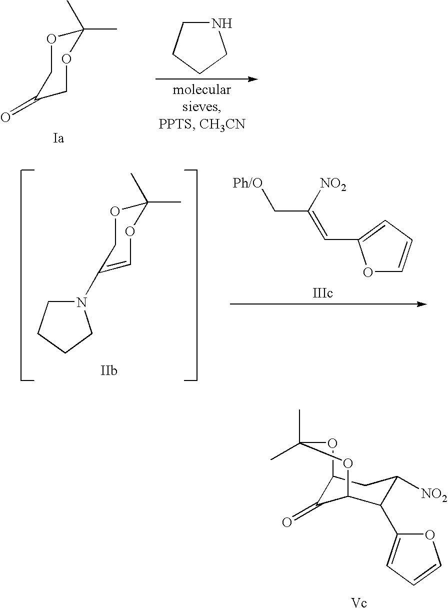

Pyrrolidine (21 μL, 0.25 mmol), molecular sieves (30 mg) and (catalytic) PPTS were added to a solution of ketone Ia (27 mg, 0.21 mmol) in acetonitrile (2 mL, 0.10 M), and the reaction mixture was stirred for 3.5 h at r.t. Nitroolefin IIIc (53 mg, 0.21 mmol) at 0° C. was added, it was left stirring for one hour at this temperature and overnight at r.t. The reaction mixture was washed with a saturated NH₄Cl solution and was extracted with CH₂Cl₂. The organic phase was dried over anhydrous Na₂SO₄ and was concentrated in a rotary evaporator. The obtained residue was purified by means of column chromatography (CH₂Cl₂-hexane 40:60), the product Vc (19 mg, 31%) being obtained. Vc data:

¹H-NMR (CDCl₃, 500 MHz, TMS) δ: 7.36 (broad s, 1H, CH), 6.36 (d, J=3.4, 1H, CH), 6.43 (dd, J=3.4, J=1.8, 1H, CH), 5.56 (ddd, J=11.7, J=11.4, J=4.9, 1H, CHNO₂), 4.49 (t, J=2.1, 1H, CH), 4.47-4.45 (m, 1H, CH), 3.81 (dd, J=11.4, J=1.5, 1H, CH), 3.01 (dt, J=13.2, J=4.9, 1H, CH₂), 2.29 (ddd, J=13.2, J=11.7, J=1.8, 1H, CH₂), 1.53 (s, 3H, CH₃), 1.45 (s, 3H, CH₃).

¹³C-NMR and DEPT (63 MHz) δ: 208.9 (CO), 149.0 (C), 142.7 (CH), 110.7 (CH), 108.3 (CH), 99.3 (C), 81.2 (CHNO₂), 77.3 (CH), 74.6 (CH), 49.9 (CH), 40.0 (CH₂), 28.4 (CH₃), 25.0 (CH₃).

### Example 6

### Preparation of Nitroolefin VIIa starting from IVa through VIa

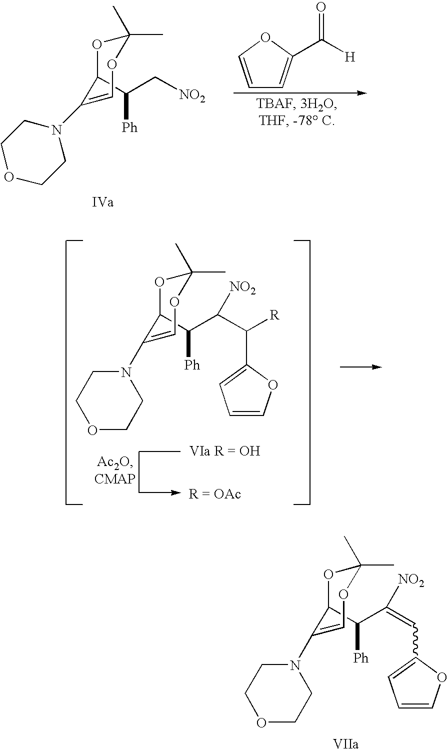

Furfural (171 μL, 2.10 mmol) and TBAF.3H₂O (734 mg, 2.26 mmol) at −78° C. were added to a solution of nitrocompound IVa (646 mg, 1.90 mmol) in dry THF (3.5 mL, 0.5 M) under argon. After 6 h of stirring, Ac₂O (218 μL, 2.26 mmol) and DMAP (69 mg, 0.56 mmol) were added. The mixture was stirred for 1.5 h at −78° C. and 12 h at r.t. The resulting reaction mixture was diluted with Et₂O and was washed with a saturated NaHCO₃ solution. The organic phase was dried over anhydrous Na₂SO₄, the solvent was evaporated in a rotary evaporator and the resulting raw product was purified by means of column chromatography (AcOEt-hexane 10:90) to obtain nitroolefin VIIa as a yellow solid (88%).

Spectroscopic Data of VIIa:

¹H-NMR (CDCl₃, 400 MHz, TMS) δ: 7.77 (s, 1H, CH), 7.65 (d, J=1.9, 1H, CH), 7.40-7.37 (m, 2H, ArH), 7.29-7.18 (m, 3H, ArH), 6.90 (d, J=3.4, 1H, CH), 6.59 (dd, J=3.4, J=1.9, 1H, CH), 6.24 (s, 1H, CH), 5.52 (d, J 8.7, 1H, CH), 5.23 (d, J=8.7, 1H, CHAr), 3.513.47 (m, 4H, CH₂), 2.85-2.77 (m, 2H, CH₂), 2.30-2.22 (m, 2H, CH₂), 1.51 (s, 3H, CH₃), 1.41 (s, 3H, CH₃).

¹³C-NMR and DEPT (100 MHz) δ: 150.0 (C), 147.8 (C), 146.1 (CH), 138.4 (C), 133.9 (CH), 131.0 (C), 128.3 (ArH), 128.0 (ArH), 126.5 (ArH), 119.9 (CH), 119.3 (CH), 112.9 (CH), 99.2 (C), 67.4 (CH), 66.3 (2×CH₂), 52.3 (2×CH₂), 48.3 (CHAr), 28.0 (CH₃), 21.8 (CH₃).

MS (Low resolution EI) m/z (%): 368 (α,β-unsaturated ketone generated by the retro-hetero-Diels-Alder reaction of the dioxenone ring, 3), 182 (77), 153 (32), 128 (30), 58 (100).

### Example 7

### Preparation of the Bicyclic Polyoxygenated Nitrogen Systems Vd and Ve

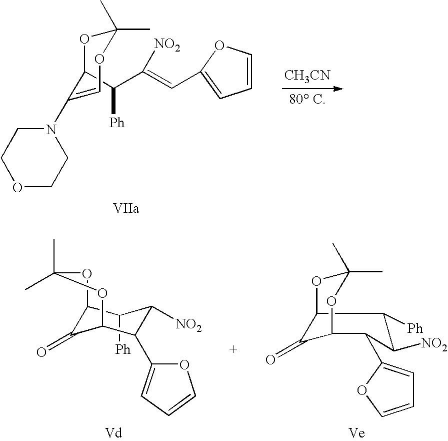

A solution of alkene VIIa (33 mg, 0.08 mmol) in acetonitrile (0.3 mL, 0.2 M) was heated at 80° C. for 30 h. The reaction mixture was concentrated in the rotary evaporator and purified by means of column chromatography (AcOEt 10:90), obtaining the bicycles Vd (6 mg, 22%) and Ve (3 mg, 10%).

Spectroscopic Data of Bicycle Ve:

¹H-NMR (CDCl₃, 250 MHz, TMS) δ: 7.36 (dd, J=1.9, J=0.9, 1H, CH), 7.32-7.27 (m, 3H, ArH), 7.18-7.14 (m, 2H, ArH), 6.28 (dd, J=3.1, J=1.9, 1H, CH), 6.23-6.21 (d, J=3.1, 1H, CH), 4.84 (dd, J=10.7, J=10.4, 1H, CHNO2), 4.61 (dd, J=10.4, J=2.5, CHAr), 4.48 (d, J=2.5, 1H, CH), 4.47 (dd, J=10.7, J=2.2, 1H, CH), 4.42-4.40 (m, 1H, CH), 1.62 (s, 3H, CH3), 1.46 (s, 3H, CH3).

¹³C-NMR and DEPT (63 MHz) δ: 207.8 (CO), 148.8 (C), 143.3 (CH), 136.8 (Ar), 129.2 (ArH), 128.4 (ArH), 127.5 (ArH), 110.5 (CH), 108.7 (CH), 101.9 (C), 87.5 (CHNO2), 78.6 (CH), 76.3 (CH), 54.6 (CHAr), 48.5 (CH), 29.7 (CH3), 25.9 (CH3).

MS (low resolution CI⁺) m/z (%): 358 (M⁺+1, 2), 252 (42), 223 (100).

IR (CsI): 1759 (intense, narrow, CO), 1561 (intense, narrow, NO₂) cm⁻¹.

### Example 8

### Preparation of Bicycles Vd and Ve Starting from IVa

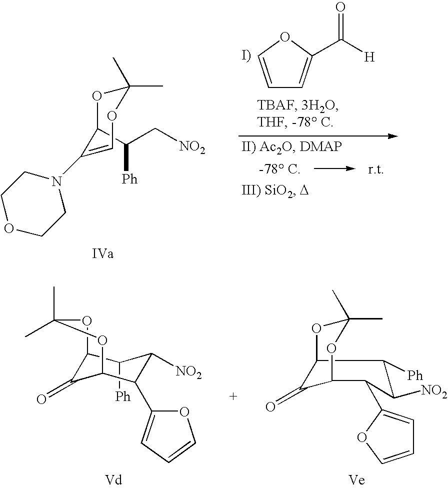

Furfural (12 μL, 0.14 mmol) and TBAF.3H₂O (54 mg, 0.16 mmol) at −78° C. were added to a solution of nitrocompound IVa (44 mg, 0.13 mmol) in dry CH₂Cl₂ (0.3 mL, 0.4 M) under argon. After 7 h of stirring, AC₂O (15 μL, 0.15 mmol) and DMAP (5 mg, 0.04 mmol) were added. The mixture was stirred for 2.5 h at −78° C. and 15 h at r.t. Then, SiO₂ (≅44 mg) was added to the reaction mixture and after 6 h under reflux, the solvent was evaporated under vacuum. Bicycles Vd (6 mg, 14%), and Ve (2 mg, 4%) were obtained after purifying the raw product by means of column chromatography (AcOEt-hexane 10:90). The starting nitrocompound IVa was partially recovered (13 mg, 30%).

### Example 9

### Preparation of Nitroolefin IIIb

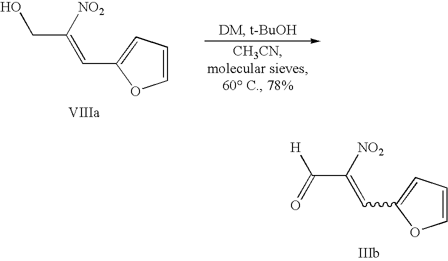

Dess Martin reagent (1.69 g, 3.98 mmol), t-butanol (562 μL, 5.97 mmol) and molecular sieves were added to a solution of alcohol VIIIa (672 mg, 3.98 mmol) in dry acetonitrile (25 mL, 0.16 M) under argon at 60° C. After half an hour, the reaction mixture was diluted with Et₂O, it was filtered and the solvent was evaporated under reduced pressure. The raw product was purified by means of a fast filtration through silica gel (AcOEt-hexane 20:80), nitroolefin IIIb being obtained as an E/Z mixture (3:2) (1.58 g, 78%).

Spectroscopic Data of the E/Z Mixture IIIb:

¹H-NMR (CDCl₃, 500 MHz, TMS) δ: 10.38 (dd, J=2.3, 1H, CH-E), 9.81 (s, 1H, CH-Z), 8.45 (d, J=3.7, 1H, CH-E), 8.36 (d, J=2.3, 1H, CH-E), 7.92 (d, J=1.6, 1H, CH-E), 7.82 (d, J=1.6, 1H, CH-Z), 7.70 (d, J=3.7, 1H, CH-Z), 7.52 (s, 1H, CH-Z), 6.77 (dd, J=3.7, J=1.6, 1H, CH-E), 6.74 (dd, J=3.7, J=1.6, 1H, CH-Z).

¹³C-NMR and DEPT (63 MHz) δ: 183.6 (CHO), 182.1 (CHO), 152.0 (CH), 150.6 (CH), 147.4 (C), 146.6 (C), 130.0 (CH), 128.3 (CH), 126.3 (CH), 125.3 (CH), 115.4 (CH), 114.9 5 (CH).

MS (low resolution EI) m/z (%): 167 (M⁺, 100), 121 (M⁺-NO₂, 2), 106 (21), 83 (24), 63 (22), 58 (100).

1. A compound having formula IV 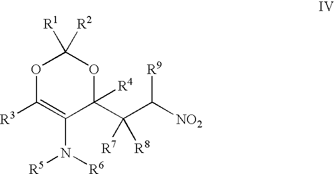
wherein R⁹ is a hydrogen or a halogen atom or a linear, branched or cyclic alkyl group, an alkenyl, aryl, heterocyclic, alkyloxy, acyloxy, aryloxy, thioalkyloxy, amino, alkylamino, arylamino, hydroxycarbonyl, alkoxycarbonyl, aryloxycarbonyl, thiocarbonyl, aminocarbonyl or hydroxy group, said groups being optionally substituted by one or several identical or different substituents selected from halogen atoms, alkyl, aryl, hydroxy, alkyloxy, acyloxy, aryloxy, amino, alkylamino, arylamino, heterocyclic, hydroxycarbonyl, alkoxycarbonyl, aryloxycarbonyl or aminocarbonyl groups, wherein R⁷ and R⁸ can have the same values as R⁹ except alkenyl, wherein R⁵ and R⁶ can be alkinyl and have the same values as R⁹ except acyloxy, hydroxycarbonyl, alkoxycarbonyl, thiocarbonyl and aminocarbonyl, wherein R³ and R⁴ can be alkinyl and have the same values as R⁹ except acyloxy, aryloxy, alkyloxy, thioalkyloxy, amino, alkylamino, arylamino and hydroxy, wherein R¹ and R² can be alkinyl and have the same values as R⁹ except amino, alkylamino, arylamino and hydroxy, wherein R¹ and R² can further jointly be a carbonyl or thiocarbonyl group, and wherein the R groups can generally be joined to one another forming cyclic structures.

2. A compound having formula V 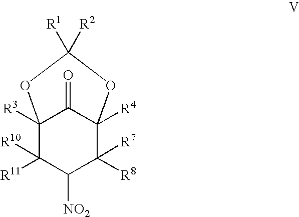
wherein the R¹, R², R³, R⁴, R⁷ and R⁸ groups can have the same values as those indicated for said groups in claim 1, wherein R¹⁰ and R¹¹ can be alkinyl and have the same values as those indicated for R⁹ in claim 1, wherein R¹⁰ and R¹¹ can further jointly be a carbonyl or thiocarbonyl group and wherein the R groups can generally be joined to one another forming cyclic structures.

3. A process for preparing compounds having formula IV characterized by the reaction of an enamine II with a nitroolefin III, wherein the R¹, R², R³, R⁴, R⁵, R⁶ , R⁷, R⁸ and R⁹ groups can have the same values as those indicated for said groups in claim 1

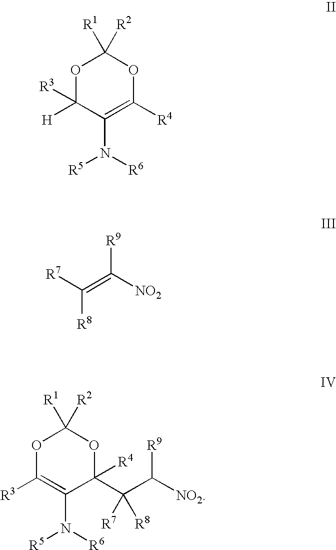

4. A process for preparing compounds having formula V characterized by the reaction of an enamine II with a nitroolefin III wherein R⁹═C(O)X wherein X can be a hydrogen or a halogen atom or O, S, Se or Te atoms joined to an alkyl, aryl or acyl group, or wherein R⁹═C[=M(CO)₅]X wherein M can be Cr, Mo or W, and X can be an O or N atom joined to one or more hydrogen atoms or to alkyl, aryl or acyl groups and wherein the R¹, R², R³, R⁴, R⁵, R⁶, R⁷, and R⁸ groups can have the same values as those indicated for said groups in claim 1 and wherein R¹⁰ and R¹¹ can be alkinyl and have the same values as those indicated for R⁹ in claim 1, wherein R¹⁰ and R¹¹ can further jointly be a carbonyl or thiocarbonyl group and wherein the R groups can generally be joined to one another forming cyclic structures:

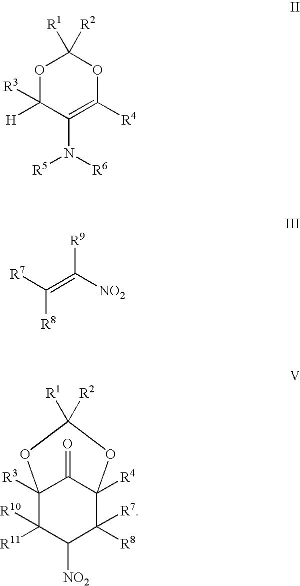

5. A process for preparing compounds having formula V according to claim 2, characterized by the reaction of an enamine II with a nitroolefin III wherein R⁹═CXYZ, wherein X and Y can be hydrogen or halogen atoms or alkyl or aryl groups which can in turn be substituted by one or several identical or different substituents selected from halogen atoms, alkyl, aryl, hydroxy, alkoxy, aryloxy, amino, alkylamino or arylamino groups, and X is any leaving group such as: a) a halogen atom, or b) an —OC(O)R^{b} group, or c) an —OS(O)ₙR^{b} wherein n can have the values 1 or 2, and R^{b} can be a linear, branched or cyclic alkyl group or an aryl group, which groups can in turn be substituted by one or several identical or different substituents selected from halogen atoms, alkyl, hydroxy, alkoxy, aryloxy, amino or alkylamino groups and wherein the R¹, R², R³, R⁴, R⁵, R⁶, R⁷, and R⁸ groups can have the same values as those indicated for said groups in claim 1 and wherein R¹⁰ and R¹¹ can be alkinyl and have the same values as those indicated for R⁹ in claim 1, wherein R¹⁰ and R¹¹ can further jointly be a carbonyl or thiocarbonyl group and wherein the R groups can generally be joined to one another forming cyclic structures:

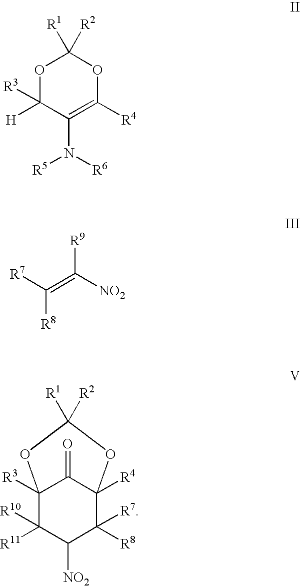

6. A process for preparing compounds having formula VI characterized by an addition reaction of a compound having formula IV to a carbonyl compound R¹⁰R¹¹(C═O), wherein the R¹, R², R³, R⁴, R⁵, R⁶, R⁷, R⁸, and R⁹ groups can have the same values as those indicated for said groups in claim 1 and wherein R¹⁰ and R¹¹ can be alkinyl and have the same values as those indicated for R⁹ in claim 1, wherein R¹⁰ and R¹¹ can further jointly be a carbonyl or thiocarbonyl group and wherein the R groups can generally be joined to one another forming cyclic structures:

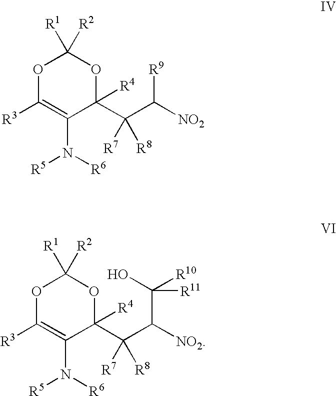

7. A process for preparing compounds having formula VII characterized by a) a conversion reaction of the hydroxy group of a compound having formula VI into a good leaving group such as a halogen atom, an SR, —OC(O)R or —OS(O)ₙR group, wherein n can have the values 1 and 2 and R can be a linear, branched or cyclic alkyl group or an aryl group, which groups can in turn be substituted by one or several identical or different substituents selected from halogen atoms, alkyl, hydroxy, alkoxy, aryloxy, amino, alkylamino or arylamino groups followed by b) the elimination of said leaving group and wherein the R¹, R², R³, R⁴, R⁵, R⁶, R⁷, R⁸, R¹⁰ and R¹¹ groups can have the same values as those indicated for said groups in claim 6

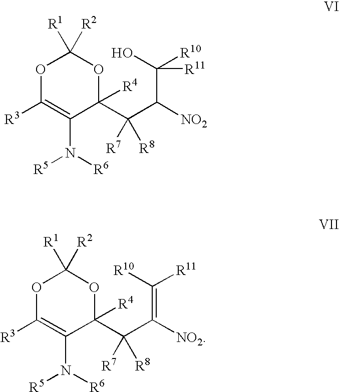

8. A process for preparing compounds having formula V characterized by the cyclization of a compound having formula VII according to claim 7, by means of an intramolecular Michael-type addition, wherein the R¹, R², R³, R⁴, R⁵, R⁶, R⁷, R⁸, and R⁹ groups can have the same values as those indicated for said groups in claim 1 and wherein R¹⁰ and R¹¹ can be alkinyl and have the same values as those indicated for R⁹ in claim 1, wherein R¹⁰ and R¹¹ can further jointly be a carbonyl or thiocarbonyl group and wherein the R groups can generally be joined to one another forming cyclic structures

9. A process for preparing nitroolefins having formula III wherein the R¹ group is a formyl group (CHO) and wherein R⁷ and R⁸ can have the same values as those indicated for said groups in claim 1, characterized by the oxidation of the corresponding precursor alcohols VIII.

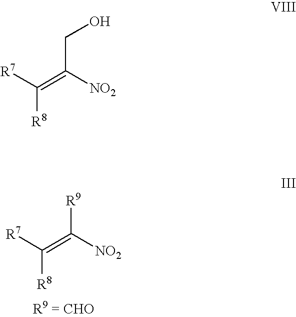

10. A pharmaceutical composition characterized in that it contains a compound having formula IV, V, VI or VII according to the previous claims as an active ingredient, either alone or forming part of a mixture with a suitable carrier or transporter.

11. A method for the manufacture of an antitumor, antiviral, antimicrobial, antifungal or anesthetic pharmaceutical composition, said method comprising use of a compound selected from among compounds of the following formulae IV, V, VI and VII: [chemical structure]
